(19) United States
(12) Patent Application Publication
(10) Pub. No.: US 2014/0350476 A1
(43) Pub. Date: Nov. 27, 2014

(54) Luer Port Alert Device And Method Of Use Thereof

(71) Applicant: Contour Fabricators, Inc., Fenton, MI (US)

(72) Inventors: Michael W. Czop, Fenton, MI (US); Clifford S. Parsons, Flushing, MI (US)

(73) Assignee: Contour Fabricators, Inc., Fenton, MI (US)

(21) Appl. No.: 14/287,362

(22) Filed: May 27, 2014

Related U.S. Application Data
(60) Provisional application No. 61/827,285, filed on May 24, 2013.

(51) Int. Cl. A61M 39/02; A61B 19/00

(52) U.S. Cl. CPC A61M 39/02; A61B 19/44; A61M 2039/0202; A61M 2039/0205
USPC 604/189

(57) ABSTRACT

A luer port alert device and method of alerting a care provider to a condition of a patient therewith prior to administering fluid treatment to the patient is provided. The luer port alert device includes an arcuate body having a through passage extending along a central axis between opposite ends. A bridge member extends from the body over one of the ends generally along the central axis to at least partially obstruct access to the through passage. The bridge member is selectively and resiliently moveable radially outwardly the central axis under an externally applied force to allow full, unobstructed access to the through passage, when desired, and returns to its relaxed position generally along the central axis upon removing the externally applied force.

### CROSS-REFERENCE TO RELATED APPLICATION

This application claims the benefit of U.S. Provisional Application Ser. No. 61/827,285, filed May 24, 2013, which is incorporated herein by reference in its entirety.

### BACKGROUND OF THE INVENTION

1. Technical Field

This invention relates generally to luer ports for operable connection to medical devices, and more particularly to devices for alerting of a patient condition at a luer port.

2. Related Art

Commonly, when in a hospital, a patient has a peripherally inserted central catheter (PICC) line inserted into a vein to facilitate injection of fluids, e.g. saline, blood, medication, or otherwise, intravenously. PICC lines typically have one or more luer ports configured for connection to medical devices, such as a luer syringe or drip bag, thereby allowing the fluid within the syringe/drip bag to be readily injected intravenously without having to locate a vein for each occurrence. The PICC line typically stays in place during the patient's stay in the hospital, and can be left in for as long as needed, and up to one year if properly maintained.

Typically, when a patient has an allergy to a medication, a bracelet is placed on the patient's wrist upon being admitted into the hospital, and the allergen is identified in the patient's chart. A potential problem associated with this method of identifying the patient allergen is that the patient's wrist band may not always be readily visible, such as when a patient drape is covering the patient's wrist, or if the wrist band falls off, and/or if the patient's chart is not updated or consulted. In addition, even if the wrist band is visible, it may not always be recognized or seen as an allergen warning or time may not be taken to read or consider the particular allergen, as conditions within a hospital often become hectic, which can interfere with the doctor or care provider from attending to or otherwise recognizing the instructions identified on the allergen band. This can prove particularly problematic during urgent care or surgery.

### SUMMARY OF THE INVENTION

In accordance with one aspect of the invention, a luer port alert device is provided. The luer port alert device includes an arcuate body having a through passage extending along a central axis between opposite ends. A bridge member extends from the body over one of the ends generally along the central axis to at least partially obstruct access to the through passage. The bridge member is selectively moveable radially outwardly the central axis to allow full, unobstructed access to the through passage.

In accordance with another aspect of the invention, the bridge member has a least one leg and a bridge portion. The at least one leg extends axially from the body in generally parallel relation with the central axis to the bridge portion and the bridge portion extends laterally from the at least one leg over the through opening.

In accordance with another aspect of the invention, the bridge member has a pair of the legs spaced on diametrically opposite sides of the arcuate body from one another.

In accordance with another aspect of the invention, the bridge portion extends in cantilevered fashion from the at least one leg to an unsupported free end.

In accordance with another aspect of the invention, the at least one leg of the bridge member is resilient.

In accordance with another aspect of the invention, at least one leg of the bridge member has a living hinge adjacent the body to facilitate movement of the bridge member radially outwardly from the central axis.

In accordance with another aspect of the invention, the luer port device has a tether strap operably attached to the body.

In accordance with another aspect of the invention, the tether strap has a resilient elongate portion extending away from the body to an annular band adapted for receipt about at least one of the luer connector and the intravenous line.

In accordance with another aspect of the invention, the annular band of the tether strap has a circumferentially discontinuous wall to facilitate coupling the annular band about at least one of the luer connector and the intravenous line.

In accordance with another aspect of the invention, the annular band of the tether strap extends about the central axis to facilitate operably coupling the luer port alert device to at least one of the luer connector and the intravenous line.

In accordance with another aspect of the invention, the elongate portion of the tether strap is generally u-shaped to further facilitate coupling the luer port alert device to at least one of the luer connector and the intravenous line.

In accordance with another aspect of the invention, the body is generally c-shaped as viewed in cross-section taken generally transversely to the central axis to further yet facilitate coupling the luer port alert device to at least one of the luer connector and the intravenous line.

In accordance with another aspect of the invention, the generally c-shaped body has a pair of fingers extending to free ends, wherein the free ends are moveable resiliently away from one another under an externally applied force.

In accordance with another aspect of the invention, a method of alerting a care provider to a condition of a patient prior to administering fluid treatment to the patient is provided. The method includes positioning a resilient bridge member over an opening of a luer connector to at least partially obstruct access to the opening, wherein the bridge member is selectively moveable from obstructing the opening to allow full, unobstructed access to the opening.

In accordance with another aspect of the invention, the method can further include tethering the bridge member to one of the luer connector and intravenous line.

In accordance with another aspect of the invention, the method can further include clipping the bridge member in selectively releasable fashion to at least one of the luer connector and intravenous line.

In accordance with another aspect of the invention, the method can further include identifying the patient's condition on the bridge member.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 is a perspective view of a luer port alert device constructed in accordance with one aspect of the invention shown operably coupled to a luer port;

FIG. 2 is a perspective view of the luer port alert device of FIG. 1 shown with a medical device operably attached to the luer port;

FIG. 3 is an enlarged view of FIG. 2;

FIG. 4 is a perspective view of the luer port alert device of FIG. 1;

FIG. 5 is a perspective view of a luer port alert device constructed in accordance with another aspect of the invention;

FIG. 6 is a perspective view of a luer port alert device constructed in accordance with yet another aspect of the invention;

FIG. 7A is a front view of the luer port alert device of FIG. 6; and

FIG. 7B is view similar to FIG. 7A with the luer port alert device shown operably coupled to a luer port with a medical device operably attached to the luer port.

### DETAILED DESCRIPTION OF PRESENTLY PREFERRED EMBODIMENTS

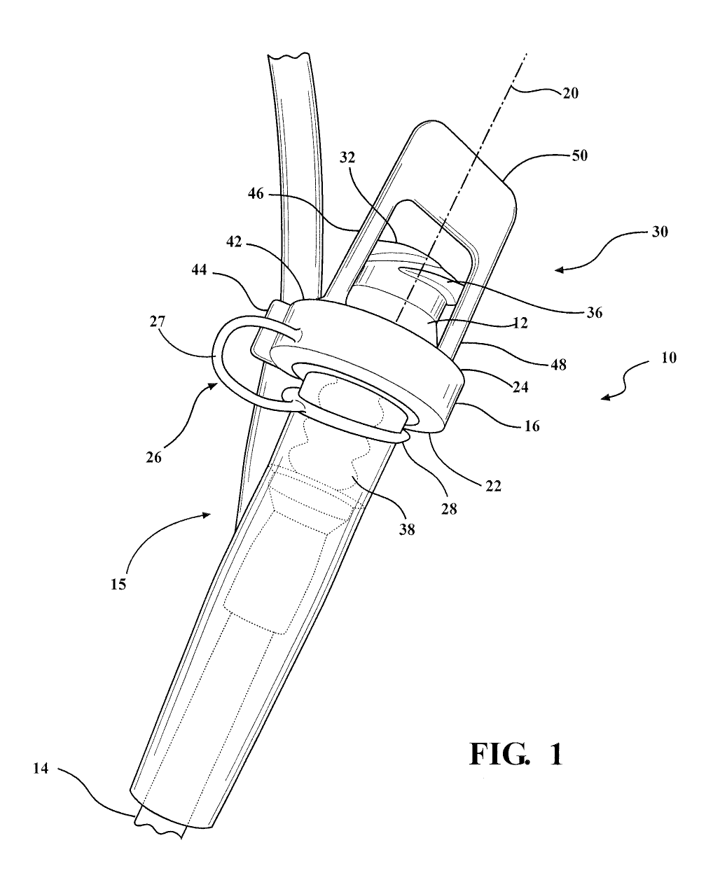

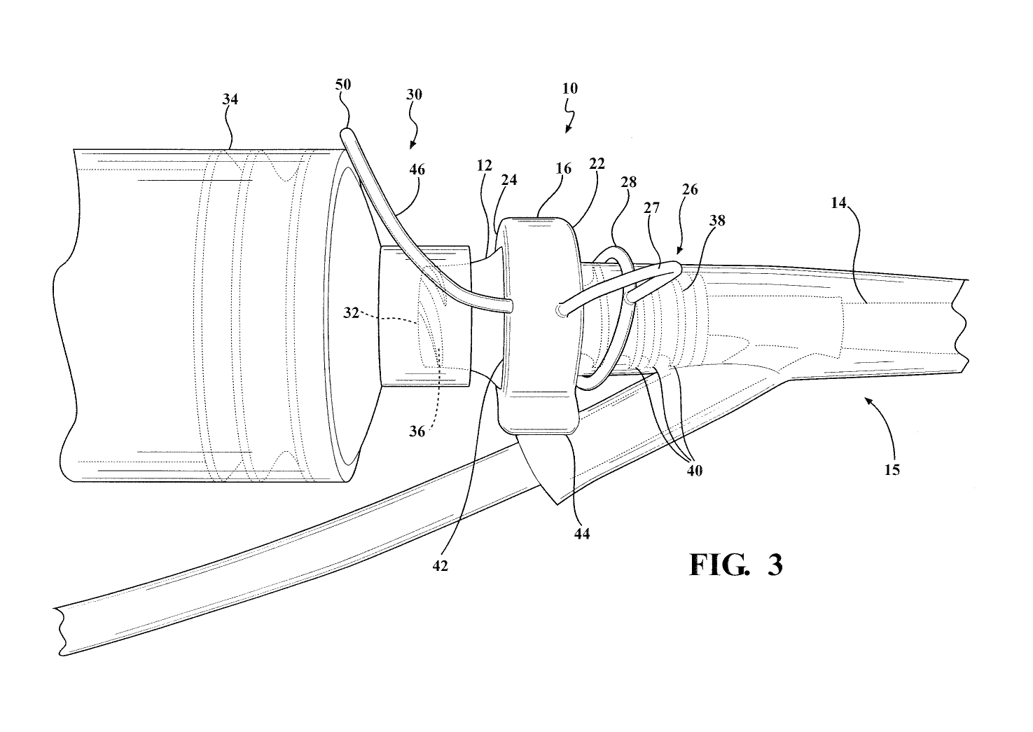

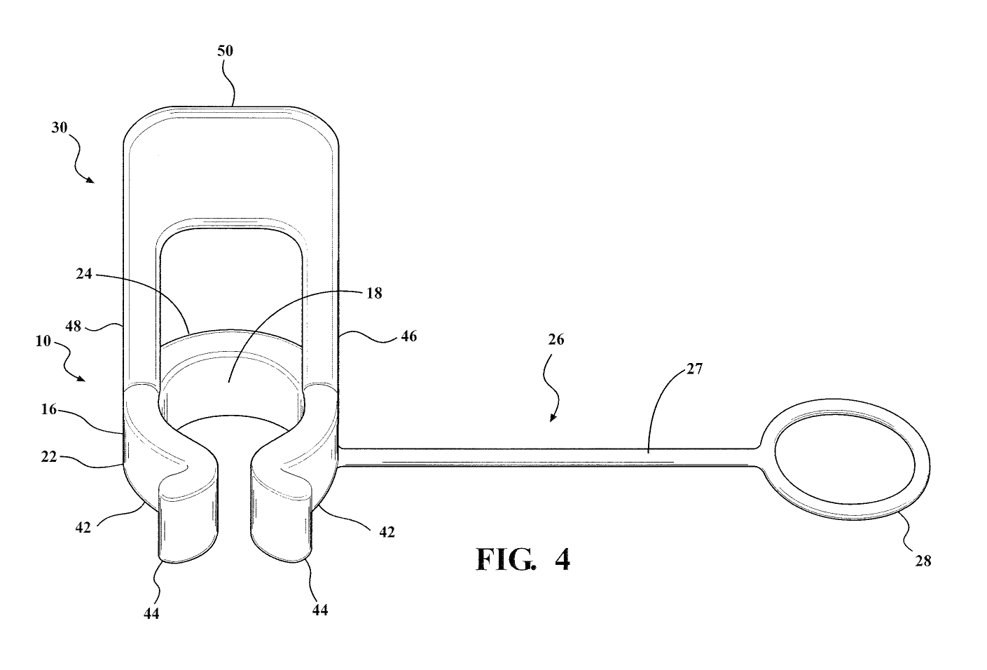

Referring in more detail to the drawings, FIGS. 1-4 illustrate a luer port alert device, referred to hereafter simply as device 10, constructed in accordance with one embodiment of the invention. The device 10 is operable for releasable attachment to at least one of a luer connector 12, an intravenous tubing or fluid line 14 and an adapter coupling sleeve 15, wherein the adaptor sleeve 15 is shown as being generally y-shaped to couple a pair of intravenous lines 14 to the luer connector 12, by way of example and without limitation. The device 10 is shown, by way of example and without limitation, as being operably coupled to both the luer connector 12 and an intravenous tubing or line 14 via the coupling sleeve 15. The device 10 includes an arcuate body 16 having a through passage 18 extending along a central axis 20 between opposite ends 22, 24. The device further includes a tether strap, referred to hereafter simply as tether 26, operably attached to the body 16, and shown, by way of example and without limitation, as extending from the body 16. The tether 26 has a resilient elongate portion 27 that extends to a free end 28 that is adapted for coupled receipt about at least one of the luer connector 12 and intravenous tubing 14, and is shown, by way of example and without limitation, as being an annular, circumferentially continuous, closed loop 28 sized for a slightly loose or line-to-line fit about at least one of the luer connector 12 and intravenous tubing 14. The device 10 has an indicator in the form of a bridge member 30 extending from the body 16 over one of the ends 24 generally along the central axis 20 to at least partially obstruct access to the luer connector 12 and through passage 18, thereby inhibiting direct, full access to the through passage 18 as well as to the luer connector 12. The bridge member 30 is selectively and resiliently moveable radially outwardly from the central axis 20 of the through passage 18 under an externally applied force to allow full, unobstructed access to the through passage 18, thereby allowing unobstructed access to the luer connector 12, when desired. As such, a care provider, in order to gain access to the luer connector 12 to administer the desired fluid treatment to the patient through the intravenous tubing 14, must intentionally deflect and bias the bridge member 30 radially outwardly from its relaxed, unbiased position along the central axis 20 to move the bridge member 30 from obstructing an opening 32 of the luer connector 12. Upon biasing the bridge member 30 radially outwardly from the central axis 20 and operably connecting a fluid treatment, such as an intravenous drip bag (not shown) or, as shown in FIGS. 3 and 4, a luer syringe 34 to the luer connector 12 and completing the injection or infusion, the care provider simply removes the luer syringe 34 from the luer connector 12, whereupon the bridge member 30 automatically springs back and returns to its relaxed, unbiased position extending generally along the central axis 20 and at least partially overlying the opening 32 of the luer connector 12. As such, the bridge member 30, upon returning to its relaxed, unbiased position, resumes its function to prevent direct access to the opening 32 of the luer connector 12, thereby continuing to act as an indicator reminding care providers of the patient's condition, such as an allergy, by way of example and without limitation, prior to their being able to administer fluids to the patient via the intravenous tubing 14.

The tubular luer connector 12 can be provided as a standard luer connector for ready attachment to the luer syringe 34, or to a connector attached to an intravenous tubing of an intravenous drip bag, for example. In the embodiment shown, the luer connector 12 joins into a standard intravenous line, such as from an IV bag, by way of example and without limitation. The luer connector 12 typically has a threaded end 36 for operable connection to the luer syringe 34 or similar mating connector attached to an intravenous line. An opposite end 38 of the luer connector 12 is typically configured for making a leak-proof connection to the intravenous line 14 via insertion into the intravenous line 14, and shown as having a plurality of annular, axially spaced retention rings 40 sized for an operable press-fit within a portion of the intravenous line 14.

The device 10 is preferably constructed as a monolithic, single piece of a resilient material, such as a molded plastic, by way of example and without limitation; however, a resilient metal material could be used, if desired. The body 16 is shown as being generally c-shaped, as viewed in cross-section taken generally transversely to the central axis 20, by way of example. As such, as best shown in FIG. 4, the body 16 has a pair of arcuate fingers 42 extending to free ends 44. The free ends 44 are moveable resiliently away from one another under an externally applied force to allow the fingers 42 to be opened and clipped or snapped about the luer connector 12 and/or intravenous line 14, and shown as the luer connector 12. To facilitate attachment of the body to the luer connector 12 and/or intravenous line 14, the free ends 44 can curl and flare laterally away from one another. To facilitate retention of the body 16, and thus the device 10, on the luer connector 12 and/or intravenous line 14, the tether 26 is operably coupled, shown as being looped, about the connector 12 and/or line 14 prior to snapping the fingers 42 about the connector 12 and/or line 14. As such, should the fingers 42 of the body 16 become inadvertently detached from their clipped position about the connector 12 and/or line 14, the tether 26 maintains the device 10 in attached relation with the connector 12 and/or line 14, thus preventing the device 10 from falling to the floor, and thereby still potentially acting to remind the care provider of the patient's condition, though not in its proper functioning position. Of course, when the care provider sees the device 10 detached from the luer connector in dangling fashion, the device 10 can be readily clipped back into its intended location.

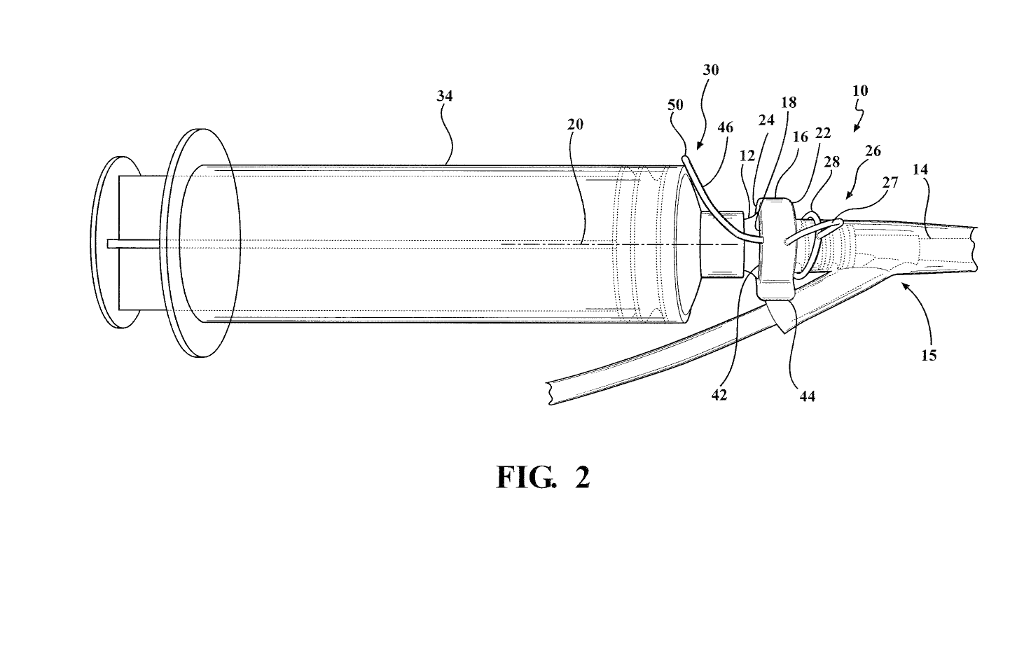

The bridge member 30 has at least one leg, and shown in the embodiment of FIGS. 1-4 as a pair of legs 46, 48, and a bridge portion 50. Each leg 46, 48 extends axially from the body 16 in generally parallel relation with the central axis 20 to the bridge portion 50. The bridge portion 50 extends laterally from and between the legs 46, 48 over the through passage 18, and thus, over the opening 32 of the luer connector 12. The legs 46, 48 are resilient, and thus, they can be intentionally deflected or biased from their relaxed, upwardly standing orientation (FIGS. 1 and 4) to a bent orientation (FIGS. 2 and 3) to selectively move the bridge portion 50 radially outwardly from the central axis 20 and the through passage 18, thereby allowing free, intended unobstructed access to the through passage 18. Being resilient, when the applied bias force is removed from the bridge portion 50, the legs 46, 48 automatically spring back to their relaxed, upstanding orientation, thereby relocating the bridge portion 50 in generally centered relation over the through passage 18 along the central axis 20 to continue acting as a reminder to the care provider of the patient's condition by at least partially obstructing access to the opening 32 of the luer connector 12. Of course, the bridge portion 50, aside from removably obstructing the opening 32 of the luer connector 32, can be labeled with the patient's condition, such as “ALLERGY”, by way of example and without limitation. Accordingly, the care provider is immediately reminded or informed, each and every time access to the luer connector 12 is needed, of the patient's condition. At the very least, the care provider is alerted by the presence of the bridge portion 50 to check the patient's charted medical information to prevent inadvertently administering an allergen, or otherwise improper treatment fluid, to the patient.

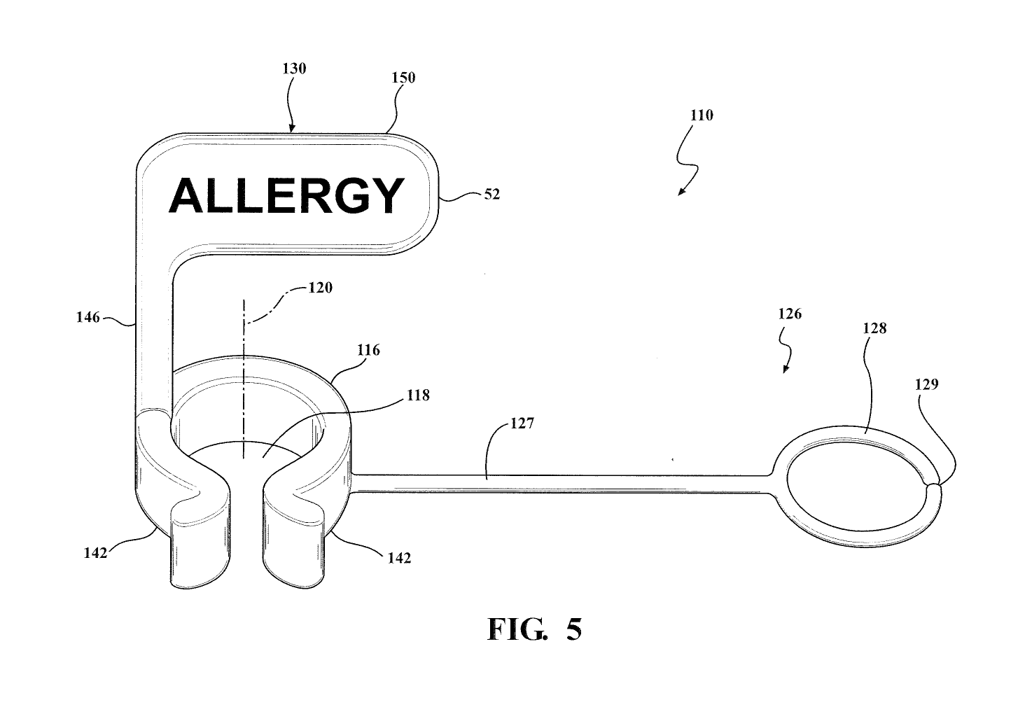

In FIG. 5, a device 110 constructed in accordance with another aspect of the invention is shown, wherein like reference numerals, offset by a factor of 100, are used to identify like features. The device 110 has a body 116 constructed generally the same as discussed above, including fingers 142 as discussed above. Further, the device 110 includes a tether 126 having a resilient elongate portion 127, generally as discussed above; however, rather than having an annular circumferentially closed and continuous free end, the device 110 has a free end 128 with a break or slit 29, thereby allowing the free end 128 to be selectively opened by opening the slit 29 to releasably dispose the annular fee end 128 about the desired fluid line or port. Another notable difference of the device 110 relative to the previously discussed device 10 is that instead of a bridge member 130 having a pair of legs, the bridge member 130 has a single leg 146 extending upwardly from the body 116 in generally parallel relation with a central axis 120 with a bridge portion 150 extending from an uppermost end of the leg 146 in cantilevered fashion over a through passage 118 of the body 116, as discussed above for the bridge portion 50 to at least partially obstruct access to the through passage 118 until selective access is desired, to an unsupported free end 52. Otherwise, the device 110 is generally the same as discussed for the device 10. Accordingly, it is to be understood that the bridge member 130 and bridge portion 150 function as discussed above for the bridge member 30 and bridge portion 50, and thus, no further discussion is necessary.

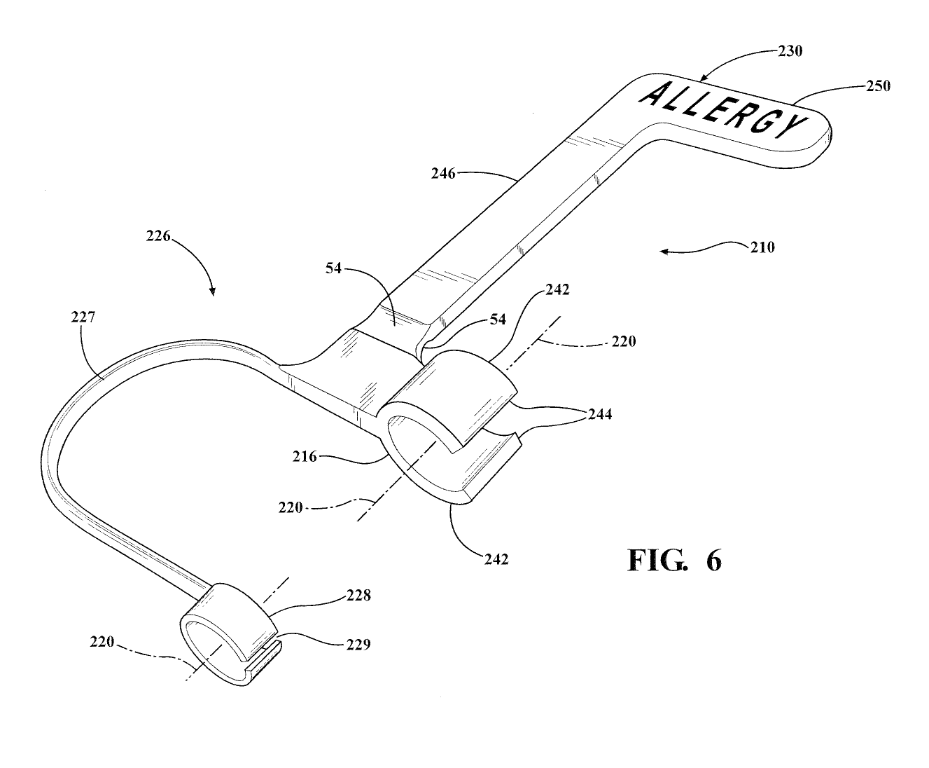

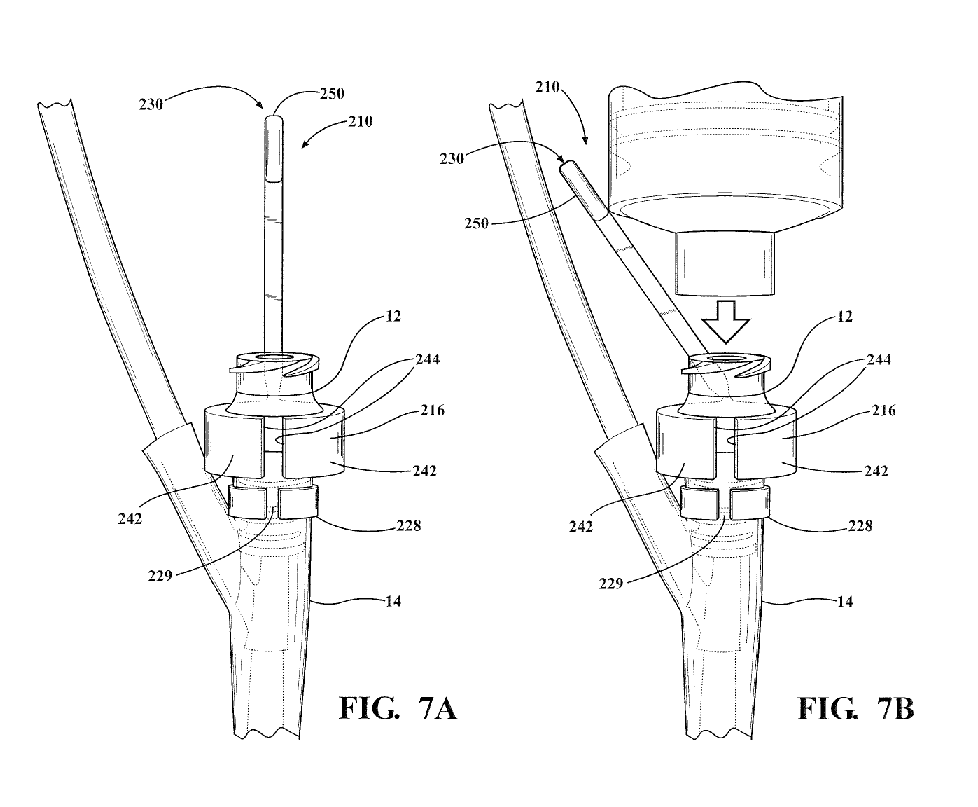

In FIGS. 6, 7A and 7B, a device 210 constructed in accordance with another aspect of the invention is shown, wherein like reference numerals, offset by a factor of 200, are used to identify like features. The device 210 has a body 216 constructed similarly as discussed above, including fingers 242 that are selectively spreadable for receipt about at least one of the luer connector 12 and/or intravenous line 14; however, rather than having free ends that flare outwardly away from one another, as discussed above for the free ends 44, the body 216 has free ends 244 that form the end of the c-shaped body 216. Of course, the c-shape of the body 216 is arced greater than 180 degrees to best ensure the body 216 remains attached in place, as desired, in use until it is desired to unclip the body 216 from the location of attachment. Further, the device 210 includes a tether 226, generally as discussed above; however, rather than having a resilient elongate portion 227 extending along a straight path from the body in a relaxed state, the resilient elongate portion 227 of the tether 226 is generally c-shaped or u-shaped in a relaxed state such that an annular or semi-annular band forming the free end 228 of the tether 226 is generally aligned with and extends about a central axis 220 of a through passage 218 of the body 216. Of course, the tether 226 is resilient, and thus, can be flexed, as desired. As with the device 110, the free end 228 has a break or slit 229, thereby allowing the free end 228 to be selectively opened by opening the slit 29 to releasably dispose the annular fee end 228 about the desired fluid line or port. As with the device 110, the device 210 has a bridge member 230 with a single leg 246 extending upwardly from the body 216 in generally parallel relation with the central axis 220 with a bridge portion 250 extending from an uppermost end of the leg 246 in cantilevered fashion over the through passage 218 of the body 216 to at least partially obstruct access to the through passage 218 until selective access is desired. However, rather than the leg 246 extending from one of the fingers 242, as shown for the devices 10, 100, the leg 246 is attached to the body 216 equidistantly between the fingers 242 and diametrically opposite the free ends 244 of the fingers 242. Further, the tether 226, rather than extending from a finger of the body 216 opposite that to which the leg 246 is attached, is operably attached to the body 216 via attachment from a base of the leg 246, wherein the arcuate u-shape of the tether 226 is generally coplanar with the body of the leg 246. As such, the device 210 is streamline and compact, given its generally flat structural configuration, which in turn, allows less space to be occupied in storage and packaging. Further, the leg 246 of the bridge member 230 has a living hinge 54 to facilitate flexing the bridge portion 250 away from the central axis 220, as desired. The living hinge 54 is shown as being formed via a necked-down, reduced thickness region of the leg 246, and is further shown as being immediately adjacent the body 216. As such, flexing of the leg 246 is enhanced to reduce the amount of force required to bend or move the leg 246 from it unflexed, upright position. Otherwise, the device 210 functions to alert care providers as discussed above for the devices 10, 110.

In accordance with another aspect of the invention, a method of alerting a care provider to a patient's condition via a device 10, 110, 210 prior to administering a fluid treatment to the patient is provided. The method includes disposing a portion 50, 150, 250 of a bridge member 30, 130, 230 of the device 10, 110, 210 over an opening 32 of a luer connector 12 connected to a fluid line 14, such as an intravenous line, to temporarily obstruct access to the opening 32, wherein the portion 50, 150, 250 of the bridge member 30, 130, 230 extending over the opening 32 is selectively and resiliently moveable from obstructing the opening 32 by the care provider to allow access to the opening. The method can further include tethering the bridge member 30, 130, 230 of the device 10, 110, 210 to one of the luer connector 12 and fluid line 14. Further yet, the method can include identifying or indicating the patient's condition on the device 10, 110, 210, such as on the bridge member 30, 130, 230. Accordingly, prior to administering the fluid treatment to the patient, the care provide is visibly and physically reminded or informed that the patient has a condition, wherein the visible and physical reminder must be selectively overridden by the care provider prior to administering treatment to the patient.

Many modifications and variations of the present invention are possible in light of the above teachings. It is, therefore, to be understood that the invention may be practiced otherwise than as specifically described, and that the scope of the invention is defined by any ultimately allowed claims.

What is claimed is:

1. A luer port alert device, comprising:
an arcuate body having a through passage extending along a central axis between opposite ends; and
a bridge member extending from said body over one of said ends generally along said central axis to at least partially obstruct access to said through passage, said bridge member being selectively moveable radially outwardly from said central axis to allow full, unobstructed access to said through passage.

2. The luer port alert device of claim 1 wherein bridge member has at least one leg and a bridge portion, said at least one leg extending axially from said body in generally parallel relation with said central axis to said bridge portion and said bridge portion extending laterally from said at least one leg over said through opening.

3. The luer port alert device of claim 2 wherein said bridge member has a pair of said legs spaced on diametrically opposite sides of said arcuate body from one another.

4. The luer port alert device of claim 2 wherein said bridge portion extends in cantilevered fashion from said at least one leg to an unsupported free end.

5. The luer port alert device of claim 2 wherein said at least one leg is resilient.

6. The luer port alert device of claim 5 wherein said at least one leg has a living hinge adjacent said body, said living hinge facilitating movement of said bridge member radially outwardly from said central axis.

7. The luer port alert device of claim 1 further including a tether operably attached to said body.

8. The luer port alert device of claim 7 wherein said tether has a resilient elongate portion extending away from said body to an annular band.

9. The luer port alert device of claim 8 wherein said annular band has a circumferentially discontinuous wall.

10. The luer port alert device of claim 9 wherein said annular band extends about said central axis.

11. The luer port alert device of claim 10 wherein said elongate portion of said tether is generally U-shaped.

12. The luer port alert device of claim 1 wherein said body has a circumferentially discontinuous wall extending about said central axis.

13. The luer port alert device of claim 12 wherein said wall is generally c-shaped as viewed in cross-section taken generally transversely to said central axis.

14. The luer port alert device of claim 12 wherein said body has a pair of arcuate fingers extending to free ends, said free ends being moveable resiliently away from one another under an externally applied force.

15. A method of alerting a care provider to a condition of a patient prior to administering fluid treatment to the patient via operable connection of the fluid treatment to a luer connector of an intravenous line, comprising:
positioning a resilient bridge member over an opening of the luer connector to at least partially obstruct access to the opening, such that the bridge member is selectively moveable from obstructing the opening to allow full, unobstructed access to the opening.

16. The method of claim 15 further including tethering the bridge member to at least one of the luer connector and intravenous line.

17. The method of claim 16 wherein the positioning further includes clipping the bridge member in selectively releasable fashion to at least one of the luer connector and intravenous line.

18. The method of claim 15 further including identifying the patient's condition on the bridge member.
